(19) United States
(12) Patent Application Publication
(10) Pub. No.: US 2012/0238914 A1
(43) Pub. Date: Sep. 20, 2012

(54) ACTIVELY CONTROLLED ORTHOTIC DEVICES

(75) Inventors: Eugene C. Goldfield, Sherborn, MA (US); Robert J. Wood, Cambridge, MA (US); Radhika Nagpal, Cambridge, MA (US); Chih-Han Yu, Taichung (TW); Leia A. Stirling, Stoneham, MA (US); Elliot Saltzman, Boston, MA (US); Dava Newman, Cambridge, MA (US)

(73) Assignees: President and Fellows of Harvard College, Cambridge, MA (US); Massachusetts Institute of Technology, Cambridge, MA (US); Trustees of Boston University, Boston, MA (US); Children's Medical Center Corporation, Boston, MA (US)

(21) Appl. No.: 13/350,031

(22) Filed: Jan. 13, 2012

Related U.S. Application Data
(63) Continuation of application No. PCT/US10/42106, filed on Jul. 15, 2010.
(60) Provisional application No. 61/225,788, filed on Jul. 15, 2009.

(51) Int. Cl. A61B 5/103; A61H 9/00

(52) U.S. Cl. 600/595; 601/151

(57) ABSTRACT

An actively controlled orthotic device includes active components that dynamically change the structural characteristics of the orthotic device according to the orientation and locomotion of the corresponding body part, or according to the changing needs of the subject over a period of use. Accordingly, the orthotic device can be effectively employed to provide locomotion assistance, gait rehabilitation, and gait training. Similarly, the orthotic device may be applied to the wrist, elbow, torso, or any other body part. The active components may be actuated to effectively transmit force to a body part, such as a limb, to assist with movement when desired. Additionally or alternatively, the active components may also be actuated to provide support of varying rigidity for the corresponding body part.

### CROSS REFERENCE TO RELATED APPLICATIONS

This application is a Continuation application of International Application No. PCT/US2010/042106 filed on Jul. 15, 2010, which claims priority to U.S. Provisional Patent Application No. 61/225,788, filed Jul. 15, 2009, the entire contents of both applications are incorporated herein by reference.

### BACKGROUND OF THE INVENTION

1. Field of the Invention

The present invention generally relates to orthotic devices, and, more particularly, to actively controlled orthotic devices having active components that can dynamically change the structural characteristics of the orthotic device according to the orientation and locomotion of the corresponding body part of the subject, or according to the changing needs of the subject over a period of use.

2. Description of Related Art

Conventional treatments of gait pathologies, such as drop-foot, spasticity, contractures, ankle equinus, crouch gait, etc., associated with neuromuscular disorders, such as cerebral palsy, may employ a passive mechanical brace to support the body parts involved in balance and gait. Depending on the severity of the gait pathology, the brace may be applied to the hip, knee, ankle, or any combination thereof to improve balance and gait and to help prevent injuries.

While passive mechanical braces may provide certain benefits, they may also lead to additional medical problems. For example, a typical treatment for preventing the foot from dragging on the ground in the case of drop-foot requires the patient to use an ankle foot orthotic (AFO). Rigid versions of the AFO constrain the ankle to a specific position, while hinged or flexible versions of the AFO allow limited plantar and dorsal flexion. By limiting the range of ankle motion, the toe can clear the ground thus allowing gait to progress more naturally and promoting increased walking speeds, increased step lengths, and reduced energy consumption during gait when compared to a subject without the device. However, the use of the AFO may result in a reduction in power generation at the ankle, as the AFO limits active plantar flexion. Additionally, the use of the AFO may lead to increased transverse-plane rotation on the knee depending on the AFO alignment. As such, the use of the AFO may yield new gait abnormalities and knee problems over time. Moreover, rigid versions of the AFO may lead to disuse atrophy of the muscles, such as the tibialis anterior muscle, potentially leading to long-term dependence on the AFO.

To address the problems caused by the rigidity of conventional orthotic devices, attempts have been made to increase the flexibility of orthotic devices and to allow a greater range of motion. However, some designs for flexible orthotic devices often fail to provide sufficient flexibility to overcome the disadvantages of a typical rigid device and to provide a desired range of motion. Moreover, although other designs of orthotic devices may provide sufficient flexibility, they generally fail to take into account the individual characteristics of the subject's movement and the subject's other possible pathological conditions. Indeed, designs for flexible orthotic devices are typically passive. As such, the devices cannot be dynamically adjusted to accommodate characteristics specific to a subject during the subject's movement. In addition, the devices cannot be dynamically adjusted to accommodate the changing needs of the subject over a period of use. In general, typical flexible orthotic devices fail to provide appropriate levels of support and assistance during the subject's movement.

### SUMMARY OF THE INVENTION

To address the deficiencies of typical orthotic devices, systems and methods according to aspects of the present invention include an actively controlled orthotic device having active components that can dynamically change the structural characteristics of the orthotic device according to the orientation and locomotion of the corresponding body part, or according to the changing needs of the subject over a period of use. Accordingly, the orthotic device according to aspects of the present invention can be effectively employed to provide locomotion assistance, gait rehabilitation, and gait training.

In one embodiment, an orthotic system includes: a garment formed from a flexible material and shaped to be worn over a body part; at least one sensor coupled to the garment, the at least one sensor providing information indicating an orientation of the body part; at least one active component incorporated with the garment, wherein in response to an actuation signal, the at least one active component changes state and causes the garment to be structurally modified; and a control system coupled to the sensor and the at least one active component, the control system being configured to receive the orientation information from the at least one sensor and provide the actuation signal to the at least one active component according to the orientation information, whereby the modification of the garment encourages a change in the orientation of the body part or provides a different level of orthotic support to the body part.

In another embodiment, an orthotic system includes: a garment formed from a flexible material and shaped to be worn over a body part; at least one active component incorporated with the garment, wherein in response to an actuation signal, the at least one active component changes state and causes the garment to be structurally modified; and a control system coupled to the at least one active component, the control system being configured to provide different actuation signals to the at least one active component over a period of use corresponding to a rehabilitation of the body part, the state of the at least one active component being modified according to the different actuation signals, whereby the garment provides different levels of assistance or support to the body part over the period of use.

A further embodiment provides a method for operating an orthotic system, the orthotic system including a garment positioned over a body part, the garment being formed from a flexible material, the method including: receiving, from at least one sensor coupled to the garment, information indicating an orientation of the body part; and in response to receiving the information from the at least one sensor, sending an actuation signal to at least one active component incorporated with the garment, wherein in response to an actuation signal, the at least one active component changes state and causes the garment to be structurally modified, whereby the modification of the garment encourages a change in the orientation of the body part or provides a different level of orthotic support to the body part.

Yet a further embodiment provides a method for operating an orthotic system, the orthotic system including a garment positioned over a body part, the garment being formed from a flexible material, the method including: receiving, from at least one sensor coupled to the garment, information indicating an orientation of the body part; and in response to receiving the information from the at least one sensor, sending different actuation signals to the at least one active component over a period of use corresponding to a rehabilitation of the body part, the state of the at least one active component being changed according to the different actuation signals, whereby the garment provides different levels of assistance or support to the body part over the period of use.

These and other aspects of the present invention will become more apparent from the following detailed description of the preferred embodiments of the present invention when viewed in conjunction with the accompanying drawings.

### BRIEF DESCRIPTION OF THE DRAWINGS

FIG. 1 illustrates an example orthotic system according to aspects of the present invention, where the orthotic system employs a garment shaped as a knee brace.

FIG. 2 illustrates a diagram of an example orthotic system according to aspects of the present invention.

FIG. 3 illustrates an example arrangement of shape memory alloy, e.g., Nitinol, wires, according to aspects of the present invention.

FIG. 4 illustrates example movement of a body part wearing a garment according aspects of the present invention.

FIG. 5A illustrates a contracted state for a pneumatic actuator, which may be employed according to aspects of the present invention.

FIG. 5B illustrates an expanded state for a pneumatic actuator, which may be employed according to aspects of the present invention.

FIG. 6 illustrates example changes in the mapping of points to the surface area about a knee when the knee changes orientation.

FIG. 7 illustrates a diagram of modules that form a garment for an orthotic system according to aspects of the present invention.

### DETAILED DESCRIPTION

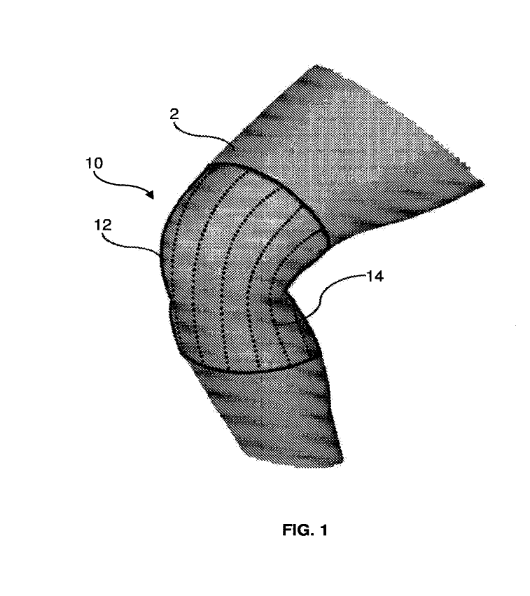

Referring to FIG. 1, an example embodiment of an orthotic system 10 according to aspects of the present invention is illustrated. In particular, FIG. 1 shows that the orthotic system 10 includes a soft, flexible garment 12 shaped as a knee brace to fit tightly over a subject's knee 2. The orthotic system 10 employs active components 14 that can be controlled to dynamically apply varying assistive and supportive contact to the subject's knee 2. In particular, the active components 14 can be embedded within, or otherwise incorporated with, the garment 12. The active components 14 can be controlled according to the orientation and locomotion of the knee 2. The orthotic system 10 can be applied to the subject's knee 2, for example, to assist in knee flexion and extension for locomotion assistance, gait rehabilitation, and gait training. Moreover, the orthotic system 10 can be used as a daily assistive device or as a rehabilitation aide.

Although the garment 12 of the orthotic system 10 is specifically shaped as a knee brace, other embodiments according to aspects of the present invention may additionally or alternatively be applied to other parts of the subject's body. For example, an embodiment may include a garment that is shaped as a sock, where the active components assist with pronation and supination in addition to plantar and dorsal flexion of the foot and ankle. The sock-shaped garment can be applied exclusively or in combination with the knee-brace-shaped garment 12 shown in FIG. 1. When the sock-shaped garment and the knee-brace-shaped garment 12 are combined, the resulting garment is shaped as a stocking and includes the active components of both the sock-shaped garment and the knee-brace-shaped garment. The active components, in this case, can be controlled according to, but not limited to, the orientation and locomotion state of the foot, shin, and thigh as well as the knee, hip, and pelvis.

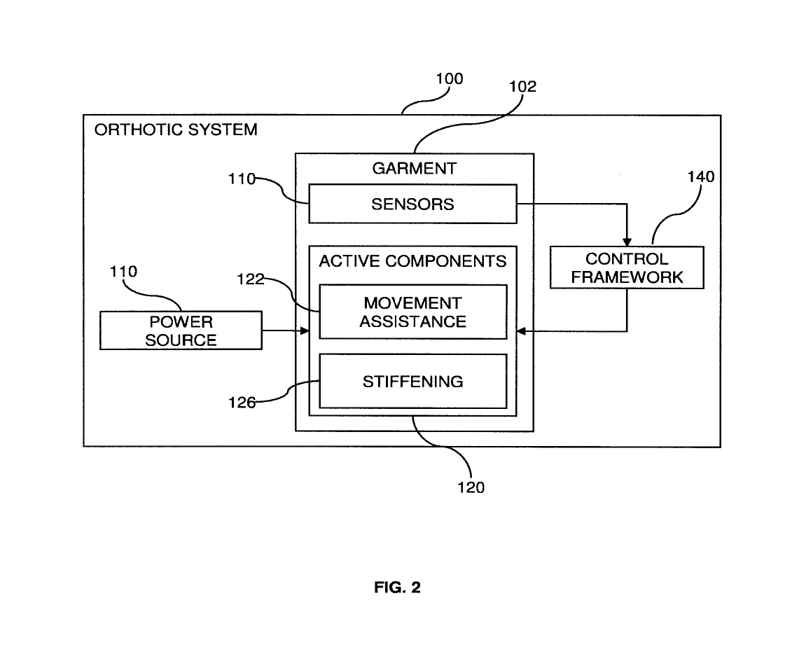

Referring to FIG. 2, further aspects of the present invention are illustrated. In particular, FIG. 2 shows an orthotic system 100 that includes sensors 110, active components 120, a portable power source 130, and a control system 140. The sensors 110 and the active components 120 can be embedded within, or otherwise incorporated with, the garment 102. In particular, the soft, flexible, tight-fitting garment 102 serves to properly position the sensors 110 and the active components 120 relative to the desired anatomical structures. These anatomical structures can include specific muscles, mechanical leverage points around joints, and/or sensory organs (e.g., muscle spindles, golgi tendon organs, etc.) which are stimulated to elicit a desired response from the sensorimotor system (e.g., reflex arcs, etc.). The knee-brace-shaped garment 12 and the active components 14 shown in FIG. 1 provide an example of how the garment 102 fits over the anatomical structures associated with the knee 2 and how the active components 120 can be positioned relative to the knee 2. However, it is understood that the orthotic system 100 can be configured for use with other body parts, such as the wrist, elbow, or torso, or with any combination of body parts.

The sensors 110 shown in FIG. 2 determine the orientation of the corresponding body part and signal this information to the control system 140. A locomotion state can be also determined or inferred from the orientation. The sensors 110 can include, but are not limited to, pressure sensors, force sensors, torque sensors, accelerometers, gyroscopes, magnetometers, strain sensors (e.g., piezoelectric polymers and carbon/elastomer composites), optical sensors, or any combination thereof.

The active components 120 include variable and adaptable materials that can be actively controlled to change the material characteristics of the garment 102 in response to changes in orientation and locomotion state. The active components 120 can be directly or indirectly connected to the portable power source 130 and the control system 140. The portable power source 130, for example, can be a portable battery pack. Meanwhile, the control system 140 can include a control board with computer processing hardware, e.g., a microprocessor, that executes programmed instructions stored on a readable storage medium, e.g., non-volatile memory. In particular, the control system 140 dynamically receives orientation information, i.e., signals, from the sensors 110, processes the signals, and actively controls the active components 120 to apply varying assistive and supportive contact to the corresponding body part.

The portable power source 130 can be attached to the garment 102 or can be carried separately on another part of the subject's body. For example, the portable power source 130 can be worn on a belt around the waist. Alternatively, the portable power source 130 may be stored in a shoe proximate to the position of the garment 102. Preferably, in some embodiments, the garment 102 is not coupled to components that are not wearable or otherwise portable. In other words, aspects of the orthotic system 100, including the battery pack 130 and the control system 140, are conveniently combined to be easily portable, and the garment is not connected by wires to a separate external computer, plug-in power supply, etc., which may prevent the subject from moving to desired locations while wearing the garment 102.

As shown further in FIG. 2, the active components 120 can be actuated to provide movement assistance 122 and/or stiffening 126. In other words, according to one aspect of the present invention, the active components 120 can be actuated to effectively transmit force to a body part, such as a limb, to assist with movement when desired. Meanwhile, according to another aspect of the present invention, the active components 120 can also be actuated to provide support of varying rigidity for the corresponding body part. Although FIG. 2 shows that the active components 120 can provide both movement assistance 122 and stiffening 126, other embodiments can include active components 120 that exclusively provide movement assistance 122 or exclusively provide stiffening 126. Although FIG. 2 may show the movement assistance 122 and stiffening 126 separately, some materials can be employed to provide both movement assistance 122 and stiffening 126.

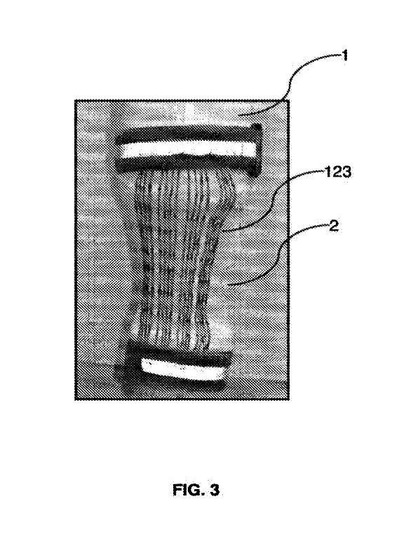

In some embodiments, the movement assistance 122 can be achieved by employing shape memory alloy wires 123 in varying arrangements as illustrated in FIG. 3. In particular, FIG. 3 shows a longitudinal arrangement of shape memory alloy wires 123, e.g., Nitinol, that can be incorporated into a garment that fits on a knee 2. When a voltage is applied to the shape memory wires 123, the wires experience a change in length. Indeed, the wires 123 in FIG. 3 are actually wound into springs to allow greater changes in length. Accordingly, the control system 140 can selectively apply voltage to particular shape memory alloy wires incorporated in the garment 102 to cause changes in length and shape for sections of the garment 102. These changes in shape may be employed to assist in desired movement of the corresponding body part. For example, the wires 123 in FIG. 3 are longitudinally aligned with a leg 1 along the back of a knee 2. As such, a shortening of the wires 123 would apply a longitudinal tension along the back of the knee 2 and cause the knee 2 to bend. Thus, the wires 123 can be controlled to assist actively with movement that involves bending of the knee.

For some materials, such as shape memory alloys, subsequent forces may be necessary to return the materials to a neutral state. In some embodiments, the orthotic system 100 can employ a configuration of opposing active components 120, for example, where the material of a particular active component is returned to a neutral state by actuating the opposing active component. For example, the wires 123 disposed along the back of the knee 2 shown in FIG. 3 can be opposed by additional shape memory alloy wires disposed along the front of the knee 2. Actuation of the wires along the front of the knee 2 causes the knee 2 to straighten rather than bend. In other cases, subsequent actuation of the same material can cause it to return to the neutral state. In yet other cases, the material can be returned to the neutral state passively, e.g., through the forces applied passively by the structure of the garment itself.

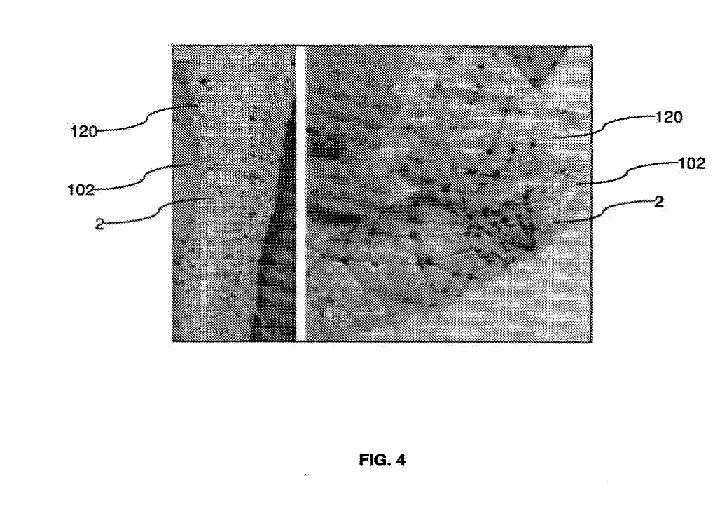

By way of example, FIG. 4 illustrates how a knee 2 has a substantially full range of movement when the active components 120, e.g., shape memory alloy wires, extending as lines in the garment 102 are in a neutral state. However, if any of the active components 120 are actuated and shortened, a reduced range of motion would occur, thus actively permitting an effective change in joint angle, i.e., knee bend.

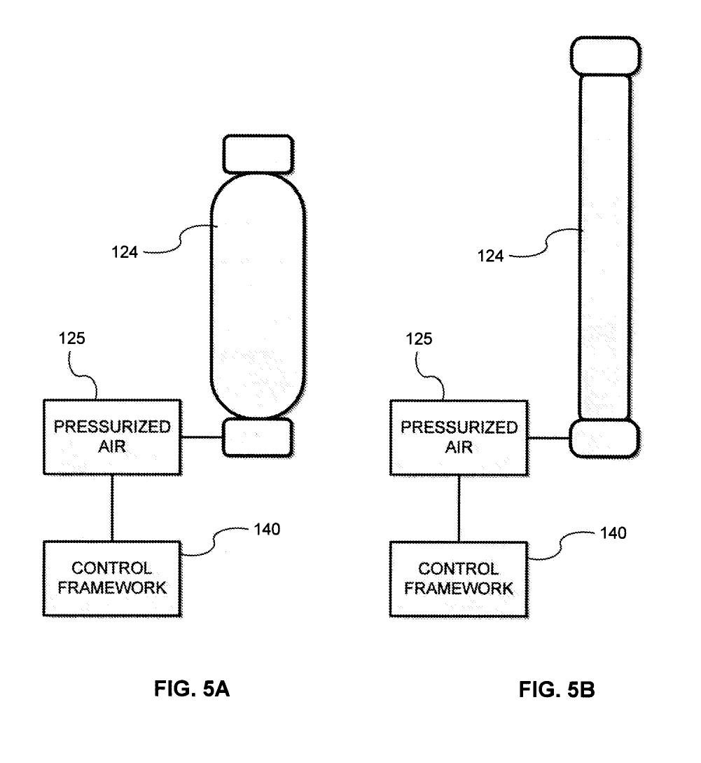

Although FIGS. 3 and 4 may illustrate the use of shape memory alloy wires 123 as active components 120, the active components 120 can employ other devices to provide movement assistance 122. In general, the active components 120 include structures that are actuated to effectively transmit force to a body part, such as a limb, to assist with movement when desired. For example, the active components 120 can employ a pneumatic actuator 124 as illustrated in FIGS. 5A-B. The control system 140 controls the amount of pressurized air 125 in the pneumatic actuator 124 to cause the pneumatic actuator 124 to change length. FIG. 5A illustrates the pneumatic actuator 124 in a contracted state, while FIG. 5B illustrates the pneumatic actuator in an expanded state. Accordingly, a plurality of pneumatic actuators 124 can be arranged in a manner similar to the shape memory alloy wires 123 shown in FIG. 3.

Thus, materials for the active components 120 can include, but are not limited to, shape memory alloys (e.g., Nitinol), shape memory polymers, ferro-fluids, magnetorheological fluids, electrorheological fluids, piezoelectric polymers, mechanochemical polymers, electroactive polymers, conductive polymers, electrostatic devices, pneumatic actuators, traditional electromagnetic devices (e.g., rotary motors and linear actuators), or any combination thereof. When actuated by the control system 140, these materials convert electrical energy as supplied by the portable power source 130 into mechanical energy.

In some embodiments, the stiffening 126 shown in FIG. 2 can be achieved by employing magnetorheological (MR) fluids, ferro-fluids, or electrorheological (ER) fluids. In particular, such fluids are enclosed within sealed capillaries within the garment 102. The sealed capillaries, for example, can be arranged longitudinally along the garment 102 in a manner similar to the shape memory alloy wires 123 shown in FIG. 3. For the MR fluids and ferro-fluids, coils of conductive wire are also positioned relative to these capillaries, providing a means to create the magnetic fields required to actuate the fluid. For the ER fluids, the electrical connection is made through embedded conductive wires. A voltage can be applied to the conductive wires to induce the alignment of the suspended particles in the fluids, thus causing an effective change in viscosity for the fluid. An increase in viscosity in a sealed capillary results in a stiffening of the garment 102 along the length of the sealed capillary. Accordingly, the control system 140 can selectively apply voltage to particular conductive wires incorporated in the garment 102 to cause changes in rigidity for sections of the garment 102 and provide support for the body part in those sections.

Accordingly, in one example, a system of sealed capillaries with MR fluids, ferro-fluids, or ER fluids and their corresponding conductive wires can be incorporated into the garment 102 to provide the stiffening 126, while a system of pneumatic actuators can be incorporated into the garment 102 to provide the motion assistance 122. In some cases the garment 102 can include multiple layers, where at least one layer includes at least one pneumatic actuator and at least one separate layer includes the stiffening capillaries. Alternatively, the pneumatic actuators and the stiffening capillaries can be incorporated into the same layer of the garment 102.

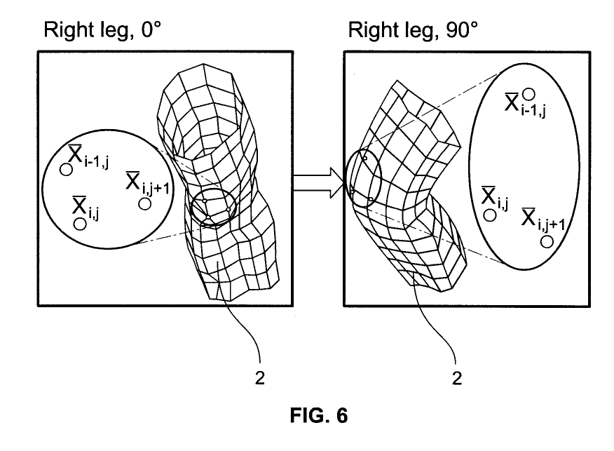

FIG. 6 shows three points mapped to a surface area about a knee. In particular, FIG. 5 shows how the relative positions of the three points change as the knee bends. Meanwhile, other points mapped to the knee 2 (not shown) may not move when the knee bends. By observing how a mapping of points changes with the movement of a body part, it is possible to determine how different areas about the body part react when the body part moves. Accordingly, it is possible to identify where the active components 120 can be applied to provide the movement assistance 122 and/or the stiffening 126 more effectively.

Although some embodiments can employ inertial measurement units, accelerometers, or the like to determine orientation of the knee, e.g., the amount of knee bend, the orientation can also be determined by identifying the relative positions of points mapped to the knee, as shown in FIG. 4. Furthermore, the amount of knee bend or changes in knee bend can indicate the knee's locomotion state, i.e., how the knee is moving. Thus, in some embodiments of the orthotic device 100, strain gauges provide a way to identify these relative positions, as strain gauges measure the relative displacement between points in a structure. In other words, strains can be correlated to orientations, such as joint angle, for a body part.

In some embodiments, the active components 120 can be coupled to the control system 140 according to separate connections, so that the control system 140 can control each active component 120 individually. As such, the control system 140 has the ability to vary the amplitude and duration of the action by each active component 120. The structural properties of each section of the garment 102 can be selectively controlled to provide the most appropriate combination of movement assistance and support for the body part in response to its orientation and locomotion at a given time. In other words, the active components 120 can be varied in stiffening and force production (amplitudes, durations) to provide effective assistance while still allowing the user to control the preferred motion and have a normal range of motion.

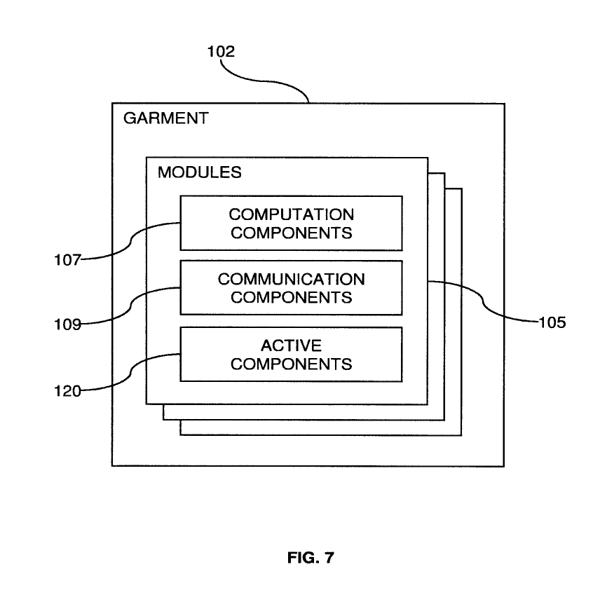

As shown in FIG. 7, the sections of the garment 102 and their corresponding active components 120 can be organized into modules, or patch regions, 105. Each module 105 is associated with an agent that coordinates with other agents to determine the most appropriate combination of assistance and support for the body part. Each module includes (1) a computation component 107 for performing computations needed in determining the appropriate actuation timing, duration, and amplitudes, (2) a communication component 109 that allows each agent to communicate with its neighbors, and (3) active components 120. The modules 105 are connected to form the flexible garment 102 that surrounds the body part, e.g., the knee, ankle, etc., and the agents coordinate with other agents to operate the combination of active components 120 simultaneously to achieve a desired time-varying task, such as preventing the toe from dragging on the ground.

With individualized control of each active component 120, the control system 140 can employ the decentralized control framework described in WIPO Publication No. WO/2009/058982 corresponding to PCT Application No. PCT/US2008/081759, filed Oct. 30, 2008 and titled ENVIRONMENTALLY-ADAPTIVE SHAPES WITH A MULTI-AGENT SYSTEM, the contents of which are incorporated entirely herein by reference. As such, the control system 140 can employ several modules that locally perform computations and control the active components 120 in a decentralized manner according to these computations. However, it is understood that the control system 140 can alternatively employ centralized control of the active components 120, where one module is responsible for performing the computations and sending a signal to all actuated components 120. The control system can activate different combinations of actuators in particular sequences, based upon sensor information about the spatial and temporal relationship of ongoing motion of the body segments.

In view of the foregoing, the orthotic device according to aspects of the present invention can be effectively employed to provide locomotion assistance, gait rehabilitation, and gait training. By providing active control in response to sensed orientation and locomotion, embodiments can take the subject's individual characteristics into account and dynamically meet the subject's individual needs. Such active control may promote more appropriate use of muscles and possibly leading to a re-education of the motor system and eventual independence from the orthotic device.

Moreover, some embodiments can provide adaptive control framework such that the level of movement assistance and stiffening provided is reduced, increased, or selectively modified over time based on the abilities of the subject as well as the progress and plan for the subject's rehabilitation and/or gait training. Indeed, aspects of the present invention can involve the use of the garment 102 as a supportive orthotic or a rehabilitative aid. When used as a supportive orthotic, for example, the garment 102 can be worn at all times when support or minor adjustment to gait is required. In this application, the control system 140 may not change the level of support over time. When used as a rehabilitative aid, for example, the garment 102 can be worn while neuromuscular function is gained or regained. In this application, however, the control system 140 can change the level of support over time.

A particular application of orthotic systems according to aspects of the present invention can focus on improving gait due to pathologies associated with cerebral palsy. However, the orthotic system can be applicable to many different mobility-impaired populations, including those with neuromuscular disorders from traumatic brain injury, loss of function due to aging or disease (e.g., MS, diabetes, etc.), or injuries, such as, those sustained during combat.

While the present invention has been described in connection with a number of exemplary embodiments, and implementations, the present inventions are not so limited, but rather cover various modifications, and equivalent arrangements.

What is claimed is:

1. An orthotic system, comprising:
a garment formed from a flexible material and shaped to be worn over a body part;
at least one sensor coupled to the garment, the at least one sensor providing information indicating an orientation of the body part;
at least one active component incorporated with the garment, wherein in response to an actuation signal, the at least one active component changes state and causes the garment to be structurally modified; and
a control system coupled to the sensor and the at least one active component, the control system being configured to receive the orientation information from the at least one sensor and provide the actuation signal to the at least one active component according to the orientation information, whereby the modification of the garment causes a change in the orientation of the body part or provides a change in a level of support to the body part.

2. The orthotic system of claim 1, wherein in response to the actuation signal, the at least one active component causes the garment to apply a force to the body part, the force causing a change in the orientation of the body part.

3. The orthotic system of claim 1, wherein in response to the actuation signal, the at least one active component changes in rigidity and causes the garment to change the level of support provided to the body part.

4. The orthotic system of claim 1, wherein the at least one sensor includes at least one of a pressure sensor, a force sensor, a torque sensor, an accelerometer, a gyroscope, a magnetometer, a strain sensor, and an optical sensor.

5. The orthotic system of claim 1, further comprising a power source coupled to the at least one active component, wherein the actuation signal includes electrical energy from the power source and the at least one active component converts the electrical energy into mechanical energy that structurally modifies the garment.

6. The orthotic system of claim 1, wherein the at least one active component includes at least one of a shape memory alloy, a shape memory polymer, a ferro-fluid, a magnetorheological fluid, an electrorheological fluid, a piezoelectric polymer, a mechanochemical polymer, an electroactive polymer, a conductive polymer, an electrostatic device, a rotary motor, a linear actuator, and a pneumatic actuator.

7. The orthotic system of claim 1, wherein the at least one active component includes at least one wire formed from a shape memory alloy, and the control system provides an actuation signal by applying a voltage to the at least one wire, the voltage causing a change in a length of the wire.

8. The orthotic system of claim 1, wherein the at least one active component includes a plurality of actuators, and at least one of the plurality of actuators is embedded in a layer of the flexible material forming the garment.

9. The orthotic system of claim 1, wherein the at least one active component includes a conductive wire and at least one sealed capillary filled with a fluid, the conductive wire being disposed along the sealed capillary, and the control system provides an actuation signal by applying a voltage to the conductive wire, the voltage causing a change in rigidity of the fluid in the sealed capillary.

10. The orthotic system of claim 9, wherein the fluid includes at least one of a ferro-fluid, a magnetorheological fluid, and an electrorheological fluid, and at least one of the sealed capillaries is embedded in a layer of the flexible material forming the garment.

11. The orthotic system of claim 1, wherein the at least one active component comprises a plurality of active components organized into modules corresponding to different sections of the garment, and the control system sends separate actuation signals to at least two of the active components.

12. The orthotic system of claim 11, wherein the control system varies the separate actuation signals to have different amplitudes and durations, the states of at least two of the active components being changed according to the different amplitudes and durations.

13. The orthotic system of claim 1, wherein the control system provides different actuation signals having different amplitudes and durations to the at least one active component over a period of use, the state of at least one active component being changed according to the different amplitudes and durations.

14. The orthotic system of claim 1, wherein the garment positions the at least one active component relative to an anatomical structure relating to gait.

15. An orthotic system, comprising:
a garment formed from a flexible material and shaped to be worn over a body part;
at least one active component incorporated with the garment, wherein in response to an actuation signal, the at least one active component changes state and causes the garment to be structurally modified; and
a control system coupled to the at least one active component, the control system being configured to provide different actuation signals to the at least one active component over a period of use corresponding to a rehabilitation of the body part, the state of the at least one active component being modified according to the different actuation signals, whereby the garment provides different levels of support to the body part over the period of use.

16. The orthotic system of claim 15, wherein in response to the actuation signal, the at least one active component causes the garment to apply a force to the body part, the force causing a change in the orientation of the body part.

17. The orthotic system of claim 15, wherein in response to the actuation signal, the at least one active component changes in rigidity and causes the garment to change the different level of support provided to the body part.

18. The orthotic system of claim 15, further comprising a power source coupled to the at least one active component, wherein the actuation signal includes electrical energy from the power source and the at least one active component converts the electrical energy into mechanical energy that structurally modifies the garment.

19. The orthotic system of claim 15, wherein the at least one active component includes at least one of a shape memory alloy, a shape memory polymer, a ferro-fluid, a magnetorheological fluid, an electrorheological fluid, a piezoelectric polymer, a mechanochemical polymer, an electroactive polymer, a conductive polymer, an electrostatic device, a rotary motor, and a linear actuators.

20. The orthotic system of claim 15, wherein the at least one active component includes at least one wire formed from a shape memory alloy, and the control system provides an actuation signal by applying a voltage to the at least one wire, the voltage causing a change in a length of the wire.

21. The orthotic system of claim 15, wherein the at least one active component includes a plurality of actuators, and at least one of the plurality of actuators is embedded in a layer of the flexible material forming the garment.

22. The orthotic system of claim 15, wherein the at least one active component includes a conductive wire and a sealed capillary filled with a fluid, the conductive wire being disposed along the sealed capillary, and the control system provides an actuation signal by applying a voltage to the conductive wire, the voltage causing a change in rigidity of the fluid in the sealed capillary.

23. The orthotic system of claim 22, wherein the fluid includes at least one of a ferro-fluid, a magnetorheological fluid, and an electrorheological fluid, and at least one of the sealed capillaries is embedded in a layer of the flexible material forming the garment.

24. The orthotic system of claim 15, wherein the at least one active component comprises a plurality of active components organized into modules corresponding to different sections of the garment, and the control system sends separate actuation signals to at least two of the active components.

25. The orthotic system of claim 15, wherein the control system varies the separate actuation signals to have different amplitudes and durations, the states of the plurality of active components being changed according to the different amplitudes and durations.

26. The orthotic system of claim 15, wherein the garment positions the at least one active component relative to an anatomical structure relating to gait.

27. A method for operating an orthotic system, the orthotic system including a garment positioned over a body part, the garment being formed from a flexible material, the method comprising:
receiving, from at least one sensor coupled to the garment, information indicating an orientation of the body part; and
in response to receiving the information from the at least one sensor, sending an actuation signal to at least one active component incorporated with the garment, wherein in response to an actuation signal, the at least one active component changes state and causes the garment to be structurally modified, whereby the modification of the garment causes a change in orientation of the body part or provides a change in a level of support to the body part.

28. The method of claim 27, wherein in response to the actuation signal, the at least one active component causes the garment to apply a force to the body part, the force causing a change in the orientation of the body part.

29. The method of claim 27, wherein in response to the actuation signal, the at least one active component changes in rigidity and causes the garment to change the level of support provided to the body part.

30. The method of claim 27, wherein the at least one sensor includes at least one of a pressure sensor, a force sensor, a torque sensor, an accelerometer, a gyroscope, a magnetometer, a strain sensor, and an optical sensor.

31. The method of claim 27, wherein sending an actuation signal includes sending electrical energy from a power source to the at least one active component, and the at least one active component converts the electrical energy into mechanical energy that structurally modifies the garment.

32. The method of claim 27, wherein the at least one active component includes at least one of a shape memory alloy, a shape memory polymer, a ferro-fluid, a magnetorheological fluid, an electrorheological fluid, a piezoelectric polymer, a mechanochemical polymer, an electroactive polymer, a conductive polymer, an electrostatic device, a rotary motor, a linear actuator, and a pneumatic actuator.

33. The method of claim 27, wherein the at least one active component includes at least one wire formed from a shape memory alloy, and sending an actuation signal to the at least one active component includes applying a voltage to at least one wire, the voltage causing a change in a length of the wire.

34. The method of claim 27, wherein the at least one active component includes a plurality of actuators, and at least one of the plurality of actuators is embedded in a layer of the flexible material forming the garment.

35. The method of claim 27, wherein the at least one active component includes a conductive wire and a sealed capillary filled with a fluid, the conductive wire being disposed along the sealed capillary, and sending an actuation signal to the at least one active component includes applying a voltage to the conductive wire, the voltage causing a change in rigidity of the fluid in the sealed capillary.

36. The method of claim 35, wherein the fluid includes at least one of a ferro-fluid, a magnetorheological fluid, or an electrorheological fluid, and at least one of sealed capillaries is embedded in a layer of the flexible material forming the garment.

37. The method of claim 27, wherein the at least one active component comprises a plurality of active components organized into modules corresponding to different sections of the garment, and sending an actuation signal to the at least one active component includes sending separate actuation signals to at least two active components.

38. The method of claim 37, wherein sending an actuation signal to the at least one active component includes varying the separate actuation signals to have different amplitudes and durations, the state of at least two active components being changed according to the different amplitudes and durations.

39. The method of claim 27, wherein sending an actuation signal to the at least one active component includes sending different actuation signals having different amplitudes and durations to the at least one active component over a period of use, the state of at least one active component being changed according to the different amplitudes and durations.

40. The method of claim 27, wherein the garment positions the at least one active component relative to an anatomical structure relating to gait.

41. A method for operating an orthotic system, the orthotic system including a garment positioned over a body part, the garment being formed from a flexible material, the method comprising:
receiving, from at least one sensor coupled to the garment, information indicating an orientation of the body part; and
in response to receiving the information from the at least one sensor, sending different actuation signals to the at least one active component over a period of use corresponding to a rehabilitation of the body part, the state of the at least one active component being changed according to the different actuation signals, whereby the garment provides different levels of support to the body part over the period of use.

42. The method of claim 41, wherein in response to the actuation signal, the at least one active component causes the garment to apply a force to the body part, the force causing a change in the orientation of the body part.

43. The method of claim 41, wherein in response to the actuation signal, the at least one active component changes in rigidity and causes the garment to change the level of support provided to the body part.

44. The method of claim 41, wherein sending different actuation signals to the at least one active component includes sending electrical energy from a power source to the at least one active component, and the at least one active component converts the electrical energy into mechanical energy that structurally modifies the garment.

45. The method of claim 41, wherein the at least one active component includes at least one of a shape memory alloy, a shape memory polymer, a ferro-fluid, a magnetorheological fluid, an electrorheological fluid, a piezoelectric polymer, a mechanochemical polymer, an electroactive polymer, a conductive polymer, an electrostatic device, a rotary motor, a linear actuator, and a pneumatic actuator.

46. The method of claim 41, wherein the at least one active component includes at least one wire formed from a shape memory alloy, and sending different actuation signals to the at least one active component includes applying a voltage to the at least one wire, the voltage causing a change in a length of the wire.

47. The method of claim 41, wherein the at least one active component includes a plurality of actuators, and at least one of the plurality of actuators is embedded in a layer of the flexible material forming the garment.

48. The method of claim 41, wherein the at least one active component includes a conductive wire and a sealed capillary filled with a fluid, the conductive wire being disposed along the sealed capillary, and sending different actuation signals to the at least one active component includes applying a voltage to the conductive wire, the voltage causing a change in rigidity of the fluid in the sealed capillary.

49. The method of claim 48, wherein the fluid includes at least one of a ferro-fluid, a magnetorheological fluid, or an electrorheological fluid, and at least one of the plurality of sealed capillaries is embedded in a layer of the flexible material forming the garment.

50. The method of claim 41, wherein the at least one active component comprises a plurality of active components organized into modules corresponding to different sections of the garment, and sending different actuation signals to the at least one active component includes sending separate actuation signals to at least two of the active components.

51. The method of claim 50, wherein sending different actuation signals to the at least one active component includes varying the separate actuation signals to have different amplitudes and durations, the states of the plurality of active components being changed according to the different amplitudes and durations.

52. The method of claim 41, wherein the garment positions the at least one active component relative to an anatomical structure relating to gait.
